(19) United States
(12) Patent Application Publication
(10) Pub. No.: US 2004/0248194 A1
(43) Pub. Date: Dec. 9, 2004

(54) Peptide screen

(76) Inventors: Carlos Garcia-Echeverria, Basel (CH); Claire Lewis, Sheffield (GB); Jeffrey Robinson, Surrey (GB)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/484,568

(22) PCT Filed: Jul. 23, 2002
(86) PCT No.: PCT/EP02/08204

(30) Foreign Application Priority Data
Jul. 23, 2001 (GB) 0117738.5
Feb. 19, 2002 (GB) 0203882.6
Feb. 19, 2002 (GB) 0203883.4

(51) Int. Cl. G01N 33/53

(52) U.S. Cl. 435/7.1

(57) ABSTRACT

The invention relates to methods to screen for agents that interact with member of the vitronectrin receptor family, αvβ3 integrin and agents obtainable by said methods.

The invention relates to methods to screen for agents that interact with a member of the vitronectin receptor family, αvβ3 integrin.

Angiogenesis, the development of new blood vessels from an existing vascular bed, is a complex multistep process that involves the degradation of components of the extracellular matrix and then the migration, proliferation and differentiation of endothelial cells to form tubules and eventually new vessels. Angiogenesis is important in normal physiological processes including, by example and not by way of limitation, embryo implantation; embryogenesis and development; and wound healing. Excessive angiogenesis is also involved in pathological conditions such as tumour cell growth and non-cancerous conditions such as neovascular glaucoma, rheumatoid arthritis, psoriasis and diabetic retinopathy.

The vascular endothelium is normally quiescent. However upon activation endothelial cells proliferate and migrate to form microtubules which will ultimately form a capillary bed to supply blood to developing tissues and, of course, a growing tumour. A number of growth factors has been identified which promote/activate endothelial cells to undergo angiogenesis. These include, by example and not by way of limitation; vascular endothelial growth factor (VEGF); transforming growth factor (TGFb); acidic and basic fibroblast growth factor (aFGF and bFGF); and platelet derived growth factor (PDGF) (1,2).

VEGF is a an endothelial cell-specific growth factor which has a very specific site of action, namely the promotion of endothelial cell proliferation, migration and differentiation. VEGF is a dimeric complex comprising two identical 23 kDa polypeptides. The monomeric form of VEGF can exist as four distinct polypeptides of different molecular weights, each being derived from an alternatively spliced mRNA. Of the four monomeric forms, two exist as membrane bound VEGF and two are soluble. VEGF is expressed by a wide variety of cell/tissue types including embryonal tissues; proliferating keratinocytes; macrophages; tumour cells. Studies (2) have shown VEGF is highly expressed in many tumour cell-lines including glioma and AIDS-associated Karposi's sarcoma VEGF activity is mediated through VEGF specific receptors expressed by endothelial cells and tumour cells. Indeed, VEGF receptors are up-regulated in endothelial cells which infiltrate tumours thereby promoting tumour cell growth.

bFGF is a growth factor which functions to stimulate the proliferation of fibroblasts and endothelial cells. bFGF is a single polypeptide chain with a molecular weight of 16.5 kDa. Several molecular forms of bFGF have been discovered which differ in the length of their amino terminal region. However the biological functions of the various molecular forms appears to be the same. bFGF is produced by the pituitary gland and is encoded by a single gene located on human chromosome 4.

A number of endogenous inhibitors of angiogenesis have been discovered, examples of which are angiostatin and endostatin, which are formed by the proteolytic cleavage of plasminogen and collagen XVIII respectively. Both of these factors have been shown to suppress the activity of pro-angiogenic growth factors such as vascular VEGF and bFGF. Both also suppress endothelial cell responses to VEGF and bFGF in vitro, and reduce the vascularisation and growth of experimental tumours in animal models.

A potent, new inhibitor of angiogenesis, which is a 50 kDa proteolytic fragment of fibrinogen, fibrinogen E, is disclosed in our co-pending application WO01/88129, which is incorporated by reference. Furthermore, we have identified a domain within the fibrinogen E fragment which has the same anti-angiogenic activity as the very much larger fibrinogen E fragment. The domain is disclosed in co-pending patent application WO02/18440, which is incorporated by reference. The domain is located at the amino terminus of the α chain and is referred to as α1-24.

We now disclose the receptor to which these polypeptides bind to mediate the anti-angiogenic effect. The peptides bind to and inhibit the activity of the vitronectin receptor, αvβ3 integrin. The vitronectin receptor is a member of a large family of receptors generally referred to as integrins.

It is known that the vitronectin receptor is involved in mediating many cellular processes. Basement membranes are organised as thin layers of specialised extracellular matrix that provide support for epithelial and endothelial cells. Basement membranes provide both mechanical support and regulate cellular behaviour such as differentiation, proliferation and migration of cells such as endothelial cells.

The αvβ3 integrin binds to numerous extracellular matrix proteins; for example, fibrinogen, fibronectin, osteopontin, thrombospondin, vitronectin and von Willebrand factor, largely via interaction with a tripeptide sequence found in the matrix proteins, arginine-glycine-aspartic acid (‘RGD’) A few other extracellular matrix proteins such as tumstatin, a collagen fragment derived from the NC1 domain of the α3 chain of collagen IV, bind to αvβ3 via a non-RGD-dependent mechanism. Maeshima et al 2000 and Maeshima et al 2001 describe a region of tumstatin which binds αvβ3 which is found between amino acids 54-132. This region does not have an RGD motif Furthermore, tumstatin binding blocks angiogenesis mediated by activated endothelial cells.

In our co-pending application (WO02/18440) we described an anti-angiogenic peptide referred to as α1-24 which lacks a canonical RGD motif and therefore binds αvβ3 in an RGD-independent fashion. The identification of this peptide which binds αvβ3 will allow the identification of agents which inhibit the binding of α1-24 and therefore identify agents with the potential to block extracellular matrix: αvβ3 interactions via this novel, non-RGD interaction pathway. The identified agents will have utility with respect to blocking angiogenesis thereby ameliorating disease conditions which depend on angiogenesis, for example wound healing, cancer.

It is an object of the invention to provide a method for the identification of agents which interfere with the interaction of a fibrinogen E fragment, or peptide derivatives thereof, and the vitronectin receptor.

It is a further object of the invention to provide a method for the identification of agents which bind the vitronectin receptor wherein the agents have angiogenesis modulatory activity.

According to an aspect of the invention there is provided a screening method for the identification of agents which modulate the interaction of a fibrinogen E fragment, or peptide derivatives thereof, with the vitronectin receptor.

In a preferred method of the invention said method comprises the steps of:

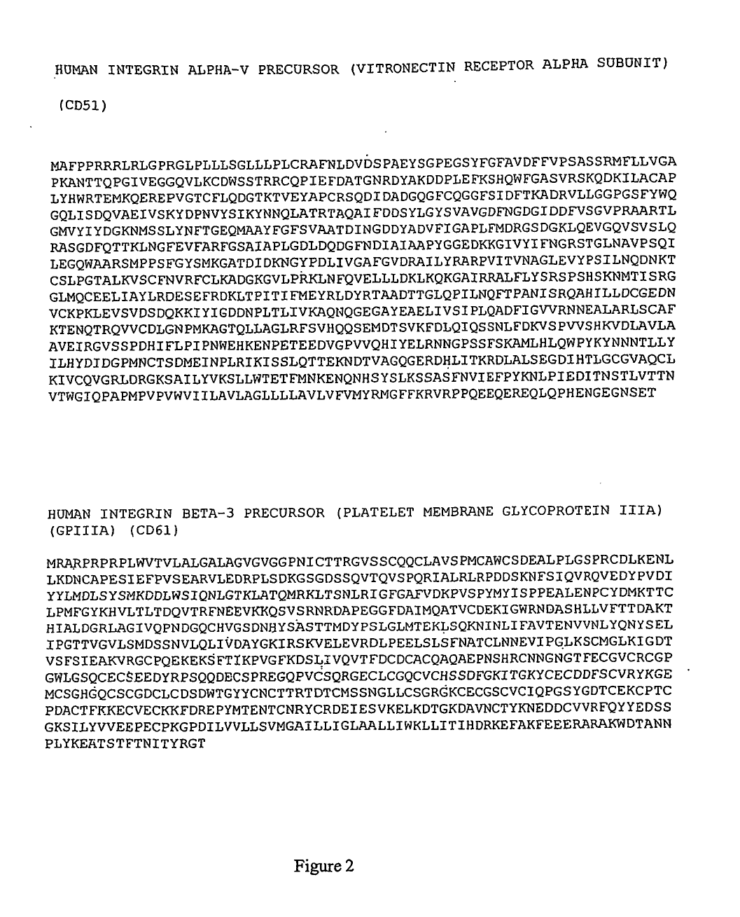

i) providing a polypeptide comprising the amino acid sequence presented in FIG. 2, or active binding fragment thereof;

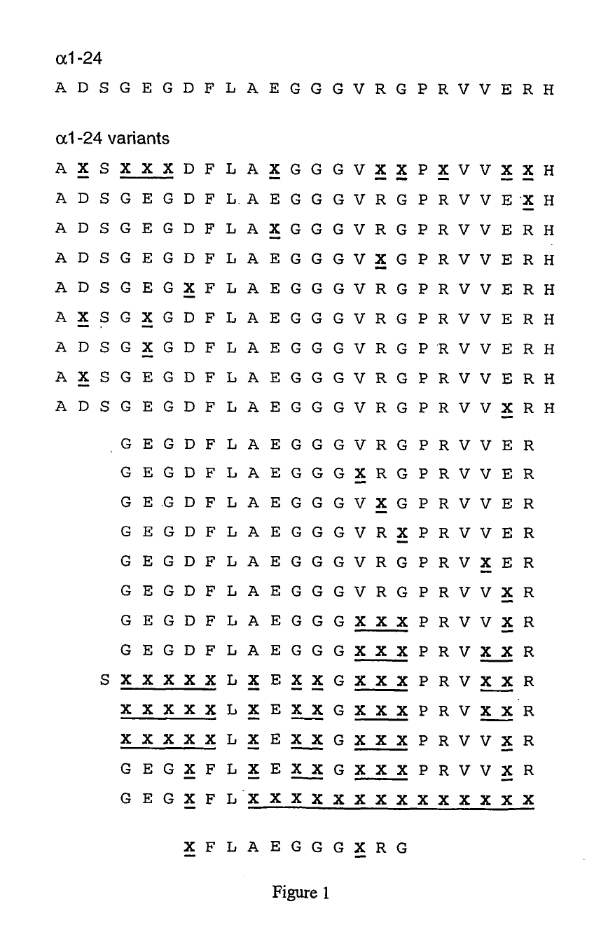

ii) providing at least one peptide comprising an amino acid sequence selected from the sequences presented in FIG. 1;

iii) providing at least one agent to be tested;

iv) forming a preparation of (i), (ii) and (iii); and

v) detecting or measuring the effect of the agent in (iii) on the interaction of the polypeptide and peptide in (i) and (ii).

In a further preferred method of the invention said agent is pre-incubated with polypeptide in (i) prior to addition of the peptide in (ii).

In an alternative preferred method of the invention said agent is pre-incubated with the peptide in (ii) prior to addition to the polypeptide in (i).

In a preferred method of the invention the peptide in (ii) comprises an amino acid sequence, or part thereof, consisting of the sequence:

XXXXXLXEXXGXXXPRVXXR

In a yet further preferred method of the invention the peptide in (ii) comprises an amino acid sequence, or part thereof, consisting of the sequence:

SXXXXXLXEXXGXXXPRVXXR

In a yet further preferred method of the invention said peptide comprises an amino acid sequence as represented by the sequence:

GEGXFLXEXXGXXXPRVVXR

In a yet further preferred method of the invention said peptide comprises an amino acid sequence as represented by the sequence:

GEG XFL XXX XXXXX XXXX XX.

X is any amino acid residue selected from the group consisting of alanine, valine, leucine, isoleucine, or proline. Preferably X is alanine.

In a preferred embodiment of the invention said peptide comprises an amino acid sequence as represented by the sequences presented in table 1. Preferably said peptides comprising said sequence have anti-angiogenic activity.

In yet a further preferred embodiment of the invention the peptide comprises an amino acid sequence as represented by the overlapping part of two fragments presented in table 1. In a more preferred embodiment, said peptide is derived from the overlapping part of the peptides AHI-401 and AHI-378 in table 1. Preferably, said peptide derived from the overlapping part of the peptides AHI-401 and AHI-378 comprises one additional amino acid residue at the N-terminus.

Thus, in a preferred method of the invention said peptide comprises an amino acid sequence as represented by the sequence:

XFLAEGGGVXG

X is any amino acid residue selected from the group consisting of A, R, N, D, C, E, Q, G, H, I, L, K, M, F, P, S, T, W, Y, V. Preferably X is selected from the group consisting of A, V, L, I and P, more preferably X is a basic amino acid selected from the group consisting of H, R and K, or an acidic amino acid selected from the group consisting of D and E.

In another preferred embodiment, the N-terminal X is selected from the group consisting of D and E, whereas the C-terminal X is selected from the group consisting of H, R and K, or alternatively, the N-terminal amino acid is selected from the group consisting of H, R and K, whereas the C-terminal X is selected from the group consisting of D and E. In a particularly preferred embodiment, the N-terminal X is D and the C-terminal X is R.

In a yet further preferred method of the invention said peptide comprises an amino acid sequence selected from the group consisting of:

GEG DFL AEG GGV RGP RVVE R
GEG DFL AEG GGX RGP RVVE R
GEG DFL AEG GGV XGP RVVE R
GEG DFL AEG GGV RXP RVVE R
GEG DFL AEG GGV RGP RVXE R
GEG DFL AEG GGV RGP RVVXR
GEG DFL AEG GGXXXP RVVXR
GEG DFL AEG GGXXXP RVXXR.

X is any amino acid residue selected from the group consisting of alanine, valine, leucine, isoleucine, or proline. Preferably X is alanine.

In a further preferred method of the invention said part thereof is represented by the amino acid sequence from +1 to +15 of the amino acid sequence:

SXXXXXLXEXXGXXXPRVXXR

In a further preferred method of the invention said part thereof is represented by the amino acid sequence from +6 to +21 of the amino acid sequence:

XXXXXLXEXXGXXXPRVXXR

In a yet further preferred method of the invention said part thereof is represented by the amino acid sequence +6 to +15 of the amino acid sequence

XXXXXLXEXXGXXXPRVXXR

In a further preferred method of the invention said peptide consists of the peptide amino acid sequences as herein disclosed.

In a further preferred embodiment of the invention said peptide is 10, 11, 12, 13, 14, 15, 16, 17, 18, 19, 20, 21, 22, 23 or 24 amino acid residues in length and comprises an amino acid sequence according to the invention.

In a preferred method of the invention the peptide in (ii) is selected from the group consisting of:

| | |
|---|---|
| 1 | AXSXXXDFLAXGGGVXXPXV VXXH |
| 2 | ADSGEGDFLAXGGGVRGPRV VEXH |
| 3 | ADSGEGDFLAXGGGVRGPRV VERH |
| 4 | ADSGEGDFLA EGGGVXGPRV VERH |
| 5 | ADSGEGXFLA EGGGVRGPRV VERH |
| 6 | AXSGXGDFLA EGGGVRGPRV VERH |
| 7 | ADSGXGDFLA EGGGVRGPRV VERH |
| 8 | AXSGEGDFLA EGGGVROPRV VERH |
| 9 | ADSGEGDFLA EGGGVRGPRV VXRH |

wherein X is any amino acid residue.

Preferably X is a hydrophobic amino acid residue and more preferably is selected from the group consisting of alanine, valine, leucine, isoleucine, or proline. Most preferably said amino acid residue is alanine.

In a further preferred method of the invention the vitronectin receptor is presented in a soluble form or by a cell. Preferably said cell naturally expresses the vitronectin receptor. Examples of cells which naturally express the vitronectin receptor are endothelial cells, smooth muscle cells, osteoclasts and tumour cells. Alternatively said cell does not naturally express the vitronectin receptor, in which case said cells are preferably genetically engineered to express the vitronectin receptor.

According to a further aspect of the invention there are provided agent(s) identified by the screening method according to the invention. Preferably said agent(s) interfere with the interaction of peptides or polypeptides with vitronectin receptor binding activity. Alternatively said agent promotes the interaction of polypeptides with vitronectin receptor binding activity.

In a preferred embodiment of the invention said agent is a foldamer, i.e. a polymer with a strong tendency to adopt a specific, compact conformation (15), that modulates the binding of α1-24 with the αvβ3 integrin.

In a more preferred embodiment of the invention said agent is a peptide, peptide derivative or low molecular weight agent that modulates the binding of α1-24 with the αvβ3 integrin.

In a further preferred embodiment of the invention said agent is a polypeptide. Preferably said agent is an antibody.

In a further preferred embodiment of the invention said antibody is a monoclonal antibody. Preferably said monoclonal antibody is a chimeric antibody. Alternatively said antibody is a humanised antibody.

Antibodies, also known as immunoglobulins, are protein molecules which have specificity for foreign molecules (antigens). Immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region.

The H chains of Ig molecules are of several classes, α, μ, σ, α, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

Chimeric antibodies are recombinant antibodies in which all of the V-regions of a mouse or rat antibody are combined with human antibody C-regions. Humanised antibodies are recombinant hybrid antibodies which fuse the complementarity-determining regions from a rodent antibody V-region with the framework regions from the human antibody V-regions. The C-regions from the human antibody are also used. The complementarity-determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not illicit an immune response. This results in a weaker immune response and a decrease in the clearance of the antibody.

According to another aspect of the invention there is provided a screening method for the identification of agents with vitronectin binding activity comprising the steps of:

i) providing a member of the integrin family which binds the peptide α1-24, or variant thereof;

ii) providing at least one candidate binding agent;

iii) forming a preparation comprising a combination of (i) and (ii);

iv) detecting or measuring the binding of the agent in (ii) with the polypeptide in (i); and optionally

v) testing the capability of the agent to modulate angiogenesis.

In a preferred method of the invention an agent with vitronectin receptor-binding activity has anti-angiogenic activity. Alternatively, the agent with vitronectin receptor binding activity has pro-angiogenic activity.

In a preferred method of the invention the integrin is the vitronectin receptor and is αvβ3 integrin. Preferably, the integrin is represented by the amino acid sequence as shown in FIG. 2, or fragment thereof.

In a further preferred method of the invention said vitronectin receptor is in a soluble form or is presented by a cell.

In a further preferred method of the invention said cell naturally expresses a vitronectin receptor.

The vitronectin receptor αvβ3 integrin is expressed by a variety of cell-types, for example, endothelial cells, smooth muscle cells, osteoclasts and tumour cells.

Preferably said cell is an endothelial cell.

In an alternative embodiment of the invention said cell does not naturally express a vitronectin receptor. Preferably said cell is genetically engineered to express the vitronectin receptor.

According to a further aspect of the invention there is provided an agent obtainable by the method according to the invention. Preferably the agent has anti-angiogenic activity. Alternatively, said agent has pro-angiogenic activity.

In a preferred embodiment of the invention the agent is a foldamer, i.e. a polymer with a strong tendency to adopt a specific, compact conformation (15). In a more preferred embodiment of the invention the agent is a low molecular weight compound, a peptide, peptide derivative or variant, or a polypeptide.

An embodiment of the invention will now be described, by example only, and with reference to the following figures:

FIG. 1 represents the amino acid sequence of the α1-24 polypeptide and α1-24 variants of fibrinogen E;

FIG. 2 is the amino acid sequence of a member of vitronectin receptor family, αvβ3 integrin (α chain and β chain;

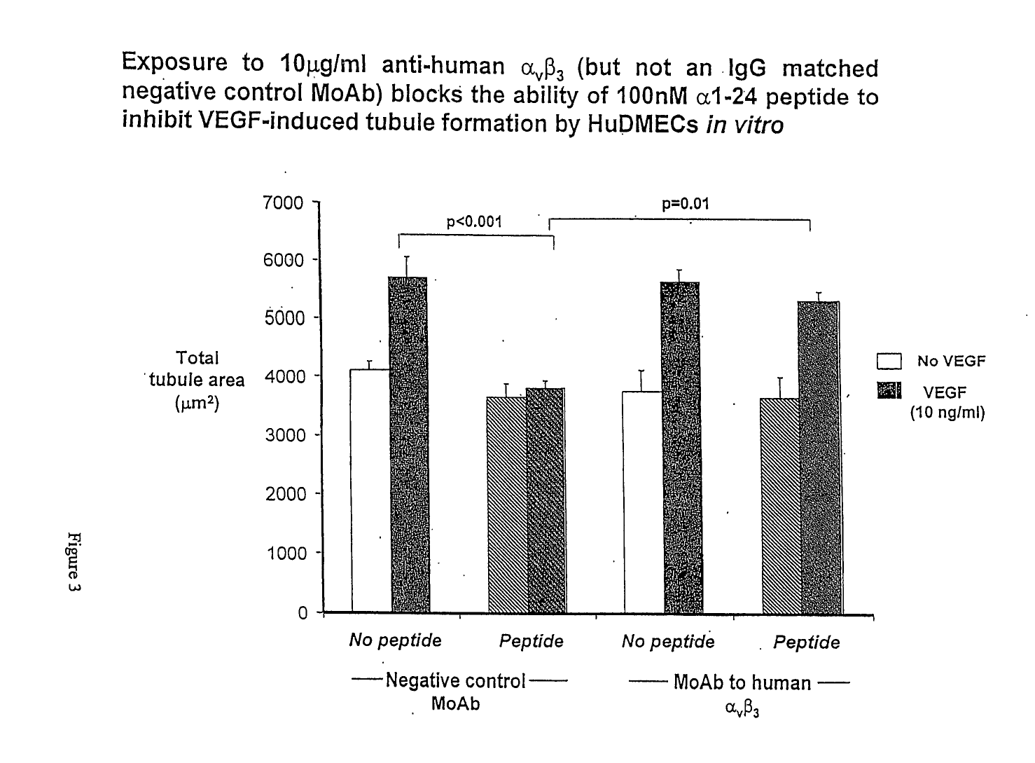

FIG. 3 represents the blocking effect of anti-vitronectin receptor polyclonal antibodies on α1-24 anti-angiogenic activity; and

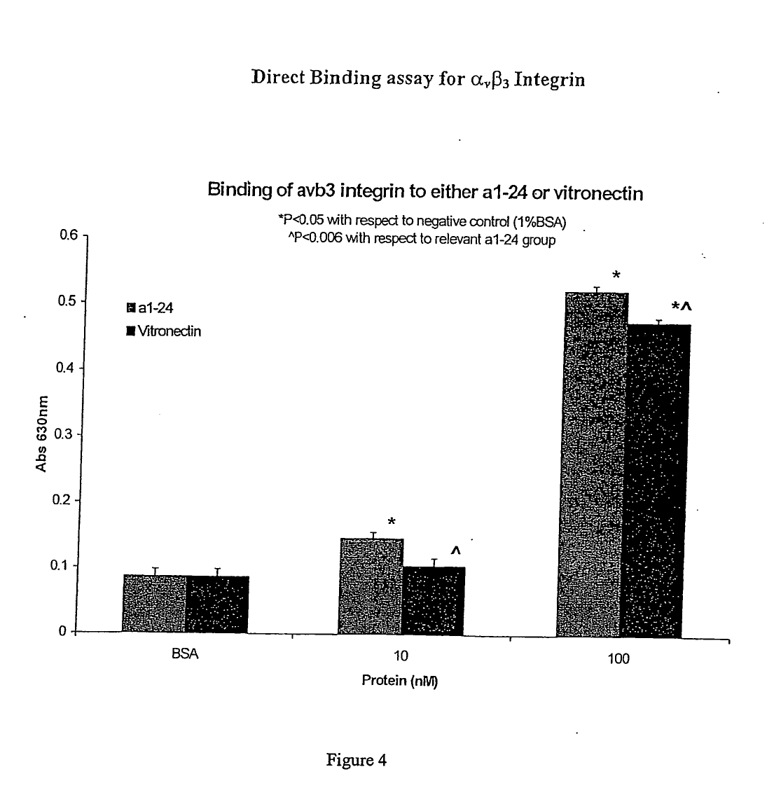

FIG. 4 represents a binding assay using immobilised. αvβ3 integrin incubated with α1-24 peptide or vitronectin.

Table 1 represents a summary of the anti-angiogenic activity of polypeptides as herein disclosed.

Materials and Methods

Human integrin αvβ3 was obtained from Chemicon International Inc (28835 Single Oak Drive Temecula, CA92590, USA, catalogue code CC1018.

Direct Binding Assay for αᵥβ₃ Integrin

1. Coat plates overnight with test protein (α1-24, modified α1-24, or vitronectin) in PBS at room temperature in sealed plates.

2. Wash three times in washing buffer (0.1% BSA, 0.05% Tween-20 in PBS)

3. Block with 1% BSA (in PBS) for 30 minutes at 37° C.

4. Wash three times with washing buffer

5. Add an equimolar amount of αᵥβ₃ integrin (Chemicon) for 1 hour at 37° C.

6. Wash three times with washing buffer

7. Add monoclonal antibody to αᵥβ₃ integrin (Chemicon) 10 μg/ml for 1 hour at 37° C.

8. Wash three times with washing buffer

9. Add secondary antibody conjugated to HRP (Dako) diluted 1:1000 in PBS for an hour at 37° C.

10. Wash three times with washing buffer

11. Add substrate—Tetramethylbenzidine (100 μg/ml) (Sigma)

12. After colour development (15-30 minutes) read on an ELISA plate reader at 630 nm.

Cell Culture.

Adult human dermal microvascular endothelial cells (HuDMECs) were obtained commercially (TCS Biologicals, Buckinghamshire, United Kingdom) and cultured in microvascular endothelial cell growth medium (EGM). This medium contains heparin (10 ng/ml), hydrocortisone, human epidermal growth factor (10 ng/ml), human fibroblast growth factor (10 ng/ml) (such endothelial growth factors are necessary for routine passaging of HuDMECs in culture) and dibutyryl cyclic AMP. This was supplemented with 5% heat-inactivated FCS, 50 μg/ml gentamicin and 50 ng/ml amphotericin B (TCS Biologicals, United Kingdom). Murine endothelial cells (SVEC 4-10) were obtained from the ATCC and cultured in DMEM+10% FCS. Cells were grown at 37° C. in a 100% humidified incubator with a gas phase of 5% CO₂ and routinely screened for mycoplasma. Prior to their use in the assays indicated below, HuDMECs were grown to 80% confluency, incubated in DMEM+1% FCS for 2 h, then harvested with 0.05% trypsin solution, washed twice and resuspended to the cell density required for each assay (see below).

α1-24 Modified Peptides.

The α1-24 peptides were generated by standard peptide synthesis methods using Fmoc amino acids and a synthesis machine. The purity of the peptide was checked by mass spectroscopy.

Tubule Formation Assay.

24 well plates were coated with 30 μl/well of growth factor-reduced (GF-reduced) Matrigel (Becton Dickinson Labware, Bedford, Mass.). Endothelial cells plated on this matrix migrate and differentiate into tubules within 6 h of plating as described previously (14). HuDMECs or SVEC 4-10 cells were seeded at a density of 4×10⁴ cells/ml and incubated for 6 h in 500 μl of either DMEM+1% FCS alone (control), or this medium ±10 ng/ml VEGF or bFGF in the presence or absence of α1-24 peptides. Assessment of tubule formation involved fixing the cell preparation in 70% ethanol at 4° C. for 15 minutes, rinsing in PBS and staining with haematoxylin and eosin. Three random fields of view in 3 replicate wells for each test condition were visualised under low power (×40 magnification), and colour images captured using a Fuji digital camera linked to a Pentium III computer (containing a frame grabber board). Tubule formation was assessed by counting the number of tubule branches and the total area covered by tubules in each field of view using image analysis software supplied by Scion Image.

Migration Assay

The Boyden chamber technique was adapted from (13) and used to evaluate HuDMEC migration across a porous membrane towards a concentration gradient of either VEGF (10 ng/ml) or bFGF (10 ng/ml). The Neuro Probe 48 well microchemotaxis chamber (Neuro Probe Inc, Cabin John, Md.) was used with 8 μm pore size polycarbonate membranes (Neuro Probe Inc, Cabin John, Md.) coated with 100 μg/ml collagen type IV. 10 ng/ml VEGF or bFGF alone or with various concentrations of α1-24 peptides were dissolved in DMEM+1% FCS and placed in the lower wells. The collagen-coated membrane was then placed over this and 50 μl of 25×10⁴ HuDMECs/ml (in DMEM containing 1% FCS) added to the upper chamber. The chambers were then incubated at 37° C. for 4.5 h. The chamber was then dismantled, the membrane removed and non-migrated cells scraped off the upper surface. Migrated cells on the lower surface were fixed with methanol, stained with Hema ‘Gurr’ rapid staining kit (Merck, Leics, United Kingdom) and counted using a light microscope (×160 magnification) in 3 random fields per well. Each test condition was carried out in 3-6 replicate wells and each experiment repeated 3 times.

Proliferation Assay

The MTT (3-[4,5-Dimethylthiazol-2-yl]-2,5-diphenyltetrazolium bromide) assay was used as previously described (12) to assess HuDMEC proliferation induced by VEGF or bFGF in the absence or presence of α1-24 peptides. HuDMEC were seeded at 3×10³ cells/100 μl in DMEM+1% FCS±10 ng/ml VEGF or bFGF in test solution into 96 well microtitre plate for 4.5 and 6 h. At these time points, a quarter volume of MTT solution (2 mg MTT/ml PBS) was added to each well and each plate was incubated for 4 h at 37° C. resulting in an insoluble purple formazan product. The medium was aspirated and the precipitates dissolved in 100 μl DMSO buffered at pH 10.5. The absorbance was then read at 540 nm on a Dynex ELISA plate reader.

Cytotoxicity Assay

HuDMECs were seeded at a density of 1-2×10⁵ cells per well in a 24 well-plate in the absence or presence of α1-24 peptides. After 6 h, both live (following removal by trysinisation) and dead (floating) cells were harvested and cell viability of all cells present assessed using propidium iodide staining of 5000 cells in each of triplicate samples per treatment using a FACScan (Becton Dickinson) equipped with a blue laser excitation of 15 mW at 488 nm. The data was collected and analysed using Cell Quest software (Becton Dickinson).

Tumour Cell Culture

The CT26 cell fine was maintained by in vitro passage in Dulbecco's Minimal Eagles Medium containing 10% foetal calf serum, and 1% penicillin and streptomycin and maintained at 37° C. in humidified atmosphere of 5% CO₂ in air. The cell line was routinely checked to ensure freedom from mycoplasma (Mycoplasma rapid detection system, Gena-Probe Incorporated, U.S.A.)

### REFERENCES

1. Folkman J Angiogenesis in cancer, vascular, rheumatoid and other disease. Nature Medicine, 1: 27-31, 1995.

2. Leek R, Harris AL, and Lewis CE Cytokine networks in solid human tumours: regulation of angiogenesis. J. Leuk. Biol., 56: 423-35, 1994.

3. Cao Y Endogenous angiogenesis inhibitors: angiostatin, endostatin, and other proteolytic fragments. Prog Mol Subcell Biol., 20:161-76, 1998.

4. Doolittle R Fibrinogen and Fibrin. Scientific American, 245: 92-101, 1981.

5. Costantini V, Zacharski L R, Memoli V A, Kisiel W, Kudryk B J, and Rousseau S M Fibrinogen deposition without thrombin generation in primary human breast cancer tissue. Cancer Res., 51:349-53, 1991.

6. Dvorak H F, Nagy J A, Feng D, Brown L F, and Dvorak A M Vascular permeability factor/vascular endothelial growth factor and the significance of microvascular hyperpermeability in angiogenesis. Curr Top Microbiol Immunol, 237:97-132, 1999.

7. Thompson W D, Wnag J E H, Wilson S J, and Ganesalingham N Angiogenesis and fibrin degradation in human breast cancer. Angiogenesis: Molecular Biology, Clinical Aspects, 245-251, 1994.

8. Thompson W D, Smith E B, Stirk C M, Marshall F I, Stout A J, and Kocchar A Angiogenic activity of fibrin degradation products is located in fibrin fragment E. J.Pathol, 168: 47-53, 1992.

9. Malinda K M, Ponce L, Kleinman H K, Shackelton L M, and Millis A J Gp38k, a protein synthesized by vascular smooth muscle cells, stimulates directional migration of human umbilical vein endothelial cells. Exp Cell Res 250:168-73, 1999.

10. Shen J, Ham R G, Karmiol S Expression of adhesion molecules in cultured human pulmonary microvascular endothelial cells. Microvasc Res., 50:360-72, 1995.

11. Liu J, Kolath J, Anderson J, Kolar C, Lawson T A, Talmadge J, and Gmeiner W H Positive interaction between 5-FU and FdUMP[10] in the inhibition of human colorectal tumour cell proliferation. Antisense Nucleic Acid Drug Dev., 9(5):481-6, 1999.

12. Dejano E, Languino L R, Polentarutti N, Balconi G, Ryckewaert J J, Larrieu M J, Donati M B, Mantovani A, and Marguerie G Interaction between fibrinogen and cultured endothelial cells. J.Clin.Invest., 75: 11-18, 1985.

13. Bootle-Wilbraham C A, Tazzyman S, Marshall J M, Lewis C E. Fibrinogen E-fragment inhibits the migration and tubule formation of human dermal microvascular endothelial cells in vitro. Cancer Research (2000) in press (Published on 1^{st} September 2000).

14. Marsh H C, Meinwald Y C, Lee S, Martinelli R A, Scheraga H A. Mechanism of action of thrombin on fibrinogen: NMR evidence for a beta-bend at or near fibrinogen A alpha Gly(P5)-Gly(P4). Biochemistry (1985) 24: 2806-2812.15.

15. Gellman S. H., Acc. Chem. Res. (1998) 31: 173-180

1. A screening for the identification of agents which modulate the interaction of the fibrinogen E fragment, or peptide derivative thereof, with a vitronectin receptor wherein said method comprises the steps of:
i) providing a polypeptide comprising the amino acid sequence presented in FIG. 2, or active binding fragment thereof;
ii) providing at least one peptide comprising an amino acid sequence selected from the sequences presented in FIG. 1;
iii) providing at least one agent to be tested;
iv) forming a preparation of (i), (ii) and (iii); and
v) detecting or measuring the effect of the agent in (iii) on the interaction of the peptide and polypeptide in (i) and (ii).

2. A method according to claim 1 wherein said agent is pre-incubated with polypeptide in (i) prior to addition of the peptide in (ii).

3. A method according to claim 1 wherein said agent is pre-incubated with the peptide in (ii) prior to addition of the agent.

4. A method according to claim 1 wherein the peptide in (ii) comprises an amino acid sequence consisting of the sequence:
XXXXXLXEXXGXXXPRVXXR.

5. A method according to claim 1 wherein the peptide in (ii) comprises an amino acid sequence consisting of the sequence:
SXXXXXLXEXXGXXXPRVXXR.

6. A method according to claim 1 wherein the peptide in (ii) comprises an amino acid sequence consisting of the sequence:
XXXXXLXEXXGXXXPRVVXR.

7. A method according to claim 1 wherein the peptide in (ii) comprises an amino acid sequence consisting of the sequence:
GEGXFLXEXXGXXXPRVVXR.

8. A method according to claim 7 wherein the peptide in (ii) comprises an amino acid sequence selected from the group consisting of:
GEGDFLAEGGGVRGPRVVER
GEGDFLAEGGGXRGPRVVER
GEGDFLAEGGGVXGPRVVER
GEGDFLAEGGGVRXPRVVER
GEGDFLAEGGGVRGPRVXER
GEGDFLAEGGGVRGPRVVXR
GEGDFLAEGGGXXXPRVVXR
GEGDFLAEGGGXXXPRVXXR
wherein X is any amino acid residue.

9. A method according to claim 1 wherein the peptide in (ii) is selected from the group consisting of:
AXSXXXDFLAXGGGVXXPXV VXXH
ADSGEGDFLAEGGGVRGPRV VEXH
ADSGEGDFLAXGGGVRGPRV VERH
ADSGEGDFLA EGGGVXGPRV VERH
ADSGEGXFLA EGGGVRGPRV VERH
AXSGXGDFLA EGGGVRGPRV VERH
ADSGXGDFLA EGGGVRGPRV VERH
AXSGEGDFLA EGGGVRGPRV VERH
ADSGEGDFLA EGGGVRGPRV VXRH
wherein X is any amino acid residue.

10. A method according to claim 1 wherein X is selected from the group consisting of alanine, valine, leucine, isoleucine, or proline.

11. A method according to claim 10 wherein X is alanine.

12. A method according to claim 1 wherein the peptide in (ii) comprises an amino acid sequence consisting of:
XFLAEGGGVXG
wherein X is any amino acid residue.

13. The method of claim 12 wherein wherein the N-terminal X is an acidic amino acid and the C-terminal X is a basic amino acid or wherein the N-terminal X is a basic amino acid and the C-terminal X is an acidic amino acid.

14. The method of claim 13 wherein wherein the N-terminal X is D and the C-terminal X is R.

15. A method according to claim 1 wherein the vitronectin receptor is soluble.

16. A method according to claim 1 wherein the vitronectin receptor is presented by a cell.

17. A method according to claim 16 wherein said cell naturally expresses the vitronectin receptor.

18. A method according to claim 16 wherein said cell is selected from the group consisting of: endothelial cells, smooth muscle cells, osteoclasts and tumour cells.

19. A method according to claim 18 wherein said cell is an endothelial cell.

20. A method according to claim 16 wherein said cell does not naturally express the vitronectin receptor.

21. A method according to claim 20 wherein said cells are genetically engineered to express the vitronectin receptor.

22. An agent(s) obtainable by the screening method according to claim 1.

23. An agent according to claim 22 wherein the agent is a foldamer, low molecular weight compound, peptide, peptide derivative or polypeptide.

24. An agent according to claim 23 wherein said agent is an antibody.

25. An agent according to claim 24 wherein said agent is a monoclonal antibody.

26. An agent according to claim 25 wherein said agent is a chimaeric antibody.

27. An agent according to claim 25 wherein said agent is a humanized antibody.

28. A screening method for the identification of agents with vitronectin binding activity which have the capability to modulate angiogenesis comprising the steps of:
i) providing a member of the integrin family which binds the peptide α1-24, or variant thereof;
ii) providing at least one candidate binding agent;
iii) forming a preparation comprising a combination of (i) and (ii);
iv) detecting or measuring the binding of the agent in (ii) with the polypeptide in (i); and optionally
iv) testing the capability of the agent to modulate angiogenesis.

29. A method according to claim 28 wherein the agent with vitronectin receptor binding activity has anti-angiogenic activity.

30. A method according to claim 28 wherein an agent with vitronectin receptor binding activity has pro-angiogenic activity.

31. A method according to claim 28 wherein the vitronectin receptor is αvβ3 integrin and comprises the amino acid sequence as represented in FIG. 2.

32. A method according to claim 28 wherein said vitronectin receptor is soluble.

33. A method according to claim 28 wherein said vitronectin receptor is presented by a cell.

34. A method according to claim 33 wherein said cell naturally expresses a vitronectin receptor.

35. A method according to claim 33 wherein said cell is selected from the group consisting of the following cell-types: endothelial cells, smooth muscle cells, osteoclasts and tumour cells.

36. A method according to claim 35 wherein said cell is an endothelial cell.

37. A method according to claim 33 wherein said cell does not naturally express vitronectin receptor.

38. A method according to claim 37 wherein said cell is genetically engineered to express the vitronectin receptor.

39. An agent obtainable by the method according to claim 28.

40. An agent according to claim 39 wherein said agent has anti-angiogenic activity.

41. An agent according to claim 39 wherein said agent has pro-angiogenic activity.

42. An agent according to claim 39 wherein the agent is a polypeptide.


SEQUENCE LISTING (76 sequences)

SEQ ID NO: 1 - protein, 24 aa, Homo sapiens
ADSGEGDFLAEGGGVRGPRVVERH

SEQ ID NO: 2 - protein, 24 aa, Homo sapiens
  misc_feature (2)..(2): X = any amino acid
AXSXXXDFLAXGGGVXXPXVVXXH

SEQ ID NO: 3 - protein, 24 aa, Homo sapiens
  MISC_FEATURE (23)..(23)
ADSGEGDFLAEGGGVRGPRVVEXH

SEQ ID NO: 4 - protein, 24 aa, Homo sapiens
  MISC_FEATURE: X = any amino acid
ADSGEGDFLAXGGGVRGPRVVERH

SEQ ID NO: 5 - protein, 24 aa, Homo sapiens
  MISC_FEATURE: X = any amino acid
ADSGEGDFLAEGGGVXGPRVVERH

SEQ ID NO: 6 - protein, 24 aa, Homo sapiens
  MISC_FEATURE: X = any amino acid
ADSGEGXFLAEGGGVRGPRVVERH

SEQ ID NO: 7 - protein, 24 aa, Homo sapiens
  MISC_FEATURE: X = any amino acid
AXSGXGDFLAEGGGVRGPRVVERH

SEQ ID NO: 8 - protein, 24 aa, Homo sapiens
  MISC_FEATURE: X = any amino acid
ADSGXGDFLAEGGGVRGPRVVERH

SEQ ID NO: 9 - protein, 24 aa, Homo sapiens
  MISC_FEATURE: X = any amino acid
AXSGEGDFLAEGGGVRGPRVVERH

SEQ ID NO: 10 - protein, 24 aa, Homo sapiens
  MISC_FEATURE: X = any amino acid
ADSGEGDFLAEGGGVRGPRVVXRH

SEQ ID NO: 11 - protein, 20 aa, Homo sapiens
  MISC_FEATURE: X = any amino acid
GEGDFLAEGGGVRGPRVVER

SEQ ID NO: 12 - protein, 20 aa, Homo sapiens
  MISC_FEATURE: X = any amino acid
GEGDFLAEGGGXRGPRVVER

SEQ ID NO: 13 - protein, 20 aa, Homo sapiens
  MISC_FEATURE: X = any amino acid
GEGDFLAEGGGVXGPRVVER

SEQ ID NO: 14 - protein, 20 aa, Homo sapiens
  MISC_FEATURE: X = any amino acid
GEGDFLAEGGGVRXPRVVER

SEQ ID NO: 15 - protein, 20 aa, Homo sapiens
  MISC_FEATURE: X = any amino acid
GEGDFLAEGGGVRGPRVXER

SEQ ID NO: 16 - protein, 20 aa, Homo sapiens
  MISC_FEATURE: X = any amino acid
GEGDFLAEGGGVRGPRVVXR

SEQ ID NO: 17 - protein, 20 aa, Homo sapiens
  MISC_FEATURE: X = any amino acid
GEGDFLAEGGGXXXPRVVXR

SEQ ID NO: 18 - protein, 20 aa, Homo sapiens
  MISC_FEATURE: X = any amino acid
GEGDFLAEGGGXXXPRVXXR

SEQ ID NO: 19 - protein, 21 aa, Homo sapiens
  MISC_FEATURE: X = any amino acid
SXXXXXLXEXXGXXXPRVXXR

SEQ ID NO: 20 - protein, 20 aa, Homo sapiens
  MISC_FEATURE: X = any amino acid
XXXXXLXEXXGXXXPRVXXR

SEQ ID NO: 21 - protein, 20 aa, Homo sapiens
  MISC_FEATURE: X = any amino acid
XXXXXLXEXXGXXXPRVVXR

SEQ ID NO: 22 - protein, 20 aa, Homo sapiens
  MISC_FEATURE: X = any amino acid
GEGXFLXEXXGXXXPRVVXR

SEQ ID NO: 23 - protein, 20 aa, Homo sapiens
  MISC_FEATURE: X = any amino acid
GEGXFLXXXXXXXXXXXXXX

SEQ ID NO: 24 - protein, 11 aa, Homo sapiens
  MISC_FEATURE: X = any amino acid
XFLAEGGGXRG

SEQ ID NO: 25 - protein, 24 aa, Homo sapiens
ADSGEGDFLAEGGGVRGPRVVERH

SEQ ID NO: 26 - protein, 24 aa, Homo sapiens
AASGEGDFLAEGGGVRGPRVVERH

SEQ ID NO: 27 - protein, 24 aa, Homo sapiens
ADAGEGDFLAEGGGVRGPRVVERH

SEQ ID NO: 28 - protein, 24 aa, Homo sapiens
ADSAEGDFLAEGGGVRGPRVVERH

SEQ ID NO: 29 - protein, 24 aa, Homo sapiens
ADSGAGDFLAEGGGVRGPRVVERH

SEQ ID NO: 30 - protein, 24 aa, Homo sapiens
ADSGEADFLAEGGGVRGPRVVERH

SEQ ID NO: 31 - protein, 24 aa, Homo sapiens
ADSGEGAFLAEGGGVRGPRVVERH

SEQ ID NO: 32 - protein, 24 aa, Homo sapiens
ADSGEGDALAEGGGVRGPRVVERH

SEQ ID NO: 33 - protein, 24 aa, Homo sapiens
ADSGEGDFAAEGGGVRGPRVVERH

SEQ ID NO: 34 - protein, 24 aa, Homo sapiens
ADSGEGDFLAAGGGVRGPRVVERH

SEQ ID NO: 35 - protein, 24 aa, Homo sapiens
ADSGEGDFLAEAGGVRGPRVVERH

SEQ ID NO: 36 - protein, 24 aa, Homo sapiens
ADSGEGDFLAEGAGVRGPRVVERH

SEQ ID NO: 37 - protein, 24 aa, Homo sapiens
ADSGEGDFLAEGGAVRGPRVVERH

SEQ ID NO: 38 - protein, 24 aa, Homo sapiens
ADSGEGDFLAEGGGARGPRVVERH

SEQ ID NO: 39 - protein, 24 aa, Homo sapiens
ADSGEGDFLAEGGGVAGPRVVERH

SEQ ID NO: 40 - protein, 24 aa, Homo sapiens
ADSGEGDFLAEGGGVRAPRVVERH

SEQ ID NO: 41 - protein, 24 aa, Homo sapiens
ADSGEGDFLAEGGGVRGARVVERH

SEQ ID NO: 42 - protein, 24 aa, Homo sapiens
ADSGEGDFLAEGGGVRGPAVVERH

SEQ ID NO: 43 - protein, 24 aa, Homo sapiens
ADSGEGDFLAEGGGVRGPRAVERH

SEQ ID NO: 44 - protein, 24 aa, Homo sapiens
ADSGEGDFLAEGGGVRGPRVAERH

SEQ ID NO: 45 - protein, 24 aa, Homo sapiens
ADSGEGDFLAEGGGVRGPRVVERH

SEQ ID NO: 46 - protein, 24 aa, Homo sapiens
ADSGEGDFLAEGGGVRGPRVVERH

SEQ ID NO: 47 - protein, 24 aa, Homo sapiens
ADSGEGDFLAEGGGVRGPRVVERH

SEQ ID NO: 48 - protein, 24 aa, Homo sapiens
ADSGEGDFLAEGGGVRGPRVVERH

SEQ ID NO: 49 - protein, 24 aa, Homo sapiens
ADSGEGDFLAEGGGVRGPRVVERH

SEQ ID NO: 50 - protein, 15 aa, Homo sapiens
ADSGEGDFLAEGGGV

SEQ ID NO: 51 - protein, 15 aa, Homo sapiens
DSGEGDFLAEGGGVR

SEQ ID NO: 52 - protein, 15 aa, Homo sapiens
SGEGDFLAEGGGVRG

SEQ ID NO: 53 - protein, 15 aa, Homo sapiens
GEGDFLAEGGGVRGP

SEQ ID NO: 54 - protein, 15 aa, Homo sapiens
EGDFLAEGGGVRGPR

SEQ ID NO: 55 - protein, 15 aa, Homo sapiens
GDFLAEGGGVRGPRV

SEQ ID NO: 56 - protein, 15 aa, Homo sapiens
DFLAEGGGVRGPRVV

SEQ ID NO: 57 - protein, 15 aa, Homo sapiens
FLAEGGGVRGPRVVE

SEQ ID NO: 58 - protein, 15 aa, Homo sapiens
LAEGGGVRGPRVVER

SEQ ID NO: 59 - protein, 15 aa, Homo sapiens
AEGGGVRGPRVVERH

SEQ ID NO: 60 - protein, 16 aa, Homo sapiens
ADSGEGDFLAEGGGVR

SEQ ID NO: 61 - protein, 28 aa, Homo sapiens
ADSGEGDFLAEGGGVRGPRVVERHQSAC

SEQ ID NO: 62 - protein, 28 aa, Homo sapiens
ADSGEGDFLAEGGGVRGPRVVERHQSAC

SEQ ID NO: 63 - protein, 23 aa, Homo sapiens
DSGEGDFLAEGGGVRGPRVVERH

SEQ ID NO: 64 - protein, 22 aa, Homo sapiens
SGEGDFLAEGGGVRGPRVVERH

SEQ ID NO: 65 - protein, 21 aa, Homo sapiens
GEGDFLAEGGGVRGPRVVERH

SEQ ID NO: 66 - protein, 20 aa, Homo sapiens
EGDFLAEGGGVRGPRVVERH

SEQ ID NO: 67 - protein, 19 aa, Homo sapiens
GDFLAEGGGVRGPRVVERH

SEQ ID NO: 68 - protein, 18 aa, Homo sapiens
DFLAEGGGVRGPRVVERH

SEQ ID NO: 69 - protein, 17 aa, Homo sapiens
FLAEGGGVRGPRVVERH

SEQ ID NO: 70 - protein, 16 aa, Homo sapiens
LAEGGGVRGPRVVERH

SEQ ID NO: 71 - protein, 15 aa, Homo sapiens
AEGGGVRGPRVVERH

SEQ ID NO: 72 - protein, 14 aa, Homo sapiens
EGGGVRGPRVVERH

SEQ ID NO: 73 - protein, 13 aa, Homo sapiens
GGGVRGPRVVERH

SEQ ID NO: 74 - protein, 12 aa, Homo sapiens
GGVRGPRVVERH

SEQ ID NO: 75 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHIFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 76 - protein, 788 aa, Homo sapiens [sequence omitted]